(19) United States
(12) Patent Application Publication
(10) Pub. No.: US 2014/0058252 A1
(43) Pub. Date: Feb. 27, 2014

(54) SINGLE HAND CONTROL DEVICE FOR ULTRASOUND GUIDED INJECTIONS

(71) Applicant: Kalle Varav, Monterey, CA (US)

(72) Inventor: Kalle Varav, Monterey, CA (US)

(21) Appl. No.: 14/009,329

(22) PCT Filed: Apr. 6, 2012
(86) PCT No.: PCT/US12/32579

§ 371 Date: Oct. 28, 2013

Related U.S. Application Data
(60) Provisional application No. 61/472,933, filed on Apr. 7, 2011.

(51) Int. Cl. A61M 5/19; A61B 8/08

(52) U.S. Cl. CPC A61M 5/19; A61B 8/0841
USPC 600/424; 604/34; 604/28

(57) ABSTRACT

Disclosed herein are various device embodiments providing for single-handed operation of dual fluid injection or fluid injection/aspiration. The inventions further contemplate methods of using the devices using real-time imaging technologies. The single-handed device has structures for selectively applying two or more sources of positive or negative pressure through an attachable needle for said dual fluid injections or aspiration.

### PRIORITY

This application claims priority to PCT Patent Application Ser. No. PCT/US2012/032579 filed Apr. 6, 2012 and to U.S. Provisional Patent Application Ser. No. 61/472,933, filed Apr. 7, 2011, both of which are incorporated herein by reference in their entirety.

### FIELD OF THE INVENTION

This invention relates to hand held devices useful for performing therapeutic and diagnostic injection and aspiration procedures with a patient. In certain instances, injection of a treatment agent is performed. In some instances, an aspiration capability is required. In other instances, sequential delivery of two or more treatment agents is performed. Generally, the device of the invention allows for the steps to be performed with a single hand held device. Further, use of the device of the present invention allows for the second hand of an operator to be able to control a real-time imaging device, such as an ultrasound transducer, to guide the injection and aspiration steps.

### BACKGROUND

The utilization of ultrasound as an image-guided tool has become common practice in medical injection procedures. The use of ultrasound allows the user to visualize anatomical structures and measure the target depth and angle during needle placement. This helps to prevent inadvertent injection into vascular or other undesired structures. In particular, in the field of anesthesiology, ultrasound guided regional anesthesia (UGRA) is rapidly becoming the standard of care. Ultrasound guidance in neural blockade procedures is being taught by most residency training programs and is a major topic of clinical research in anesthesia-focused journal publications. For hydro-dissection procedures, real-time visualization allows the user to inject small aliquots of injectate to help separate tissue planes and allow for better visualization of target structures. This tissue separation step is also commonly employed while performing UGRA procedures as it allows for better distribution of the anesthetic around the target structures.

Ultrasound guidance during injection is also useful in procedures such as joint injections, tendon or muscle injections (e.g. steroids and non-steroid agents, physiological solutions, anesthetics, and prolotherapies), deep vascular access (e.g. vein sclerotherapy), suction biopsies, aspiration of bodily fluids, automatic injection of fluid medications into the body, detection of body compartments, and intralesional injections such as with chemotherapies delivered directly in or near tumors.

The following disclosure and attached drawings describe examples of some embodiments of the invention. The designs, figures, and description are non-limiting examples. Other embodiments of the system may or may not include the features disclosed herein.

### SUMMARY OF THE INVENTION

Disclosed herein are various device embodiments providing for one-handed operation of dual fluid injection and/or aspiration and methods of using the devices. This includes, but not limited to, systems and methods for injecting and aspirating fluids into patients.

Having a single-handed injection/aspiration device that allows the physician to have sole control in a convenient grip would greatly improve image-guided procedures. In one embodiment, the single hand operated control device comprises finger actuators that regulate the injection or aspiration of fluid. The device is dimensioned so that one or more of an operator's fingers can actuate the flow of fluid by a sliding switch, scroll wheel, control buttons, or other types of actuation structures.

In certain embodiments, pressure syringes are attached to the single hand operated control device. The pressure syringes provide positive or negative pressure to inject or aspirate, respectively, fluids in response to activation of a flexible tube system by the operator.

In yet another embodiment, the single hand control device is connected to a casing that is strapped to the forearm of an operator.

In certain embodiments, the device for single-handed aspiration and injection comprises: a) an axially extending barrel having a cavity comprising a first flexible tube and a second flexible tube, said tubes extending from a proximal end to a distal end, said barrel comprising a structure for selectively pinching said first and said second flexible tubes; b) a connecting structure for converging the contents of said flexible tubes into a proximal end of a single output lumen, wherein the connecting structure is mounted on and extending distally from said barrel and wherein a fitting for a hypodermic needle is connected to a distal end of said distal connector; and c) a supporting structure on the proximal end of said barrel for connecting a first pressure delivering structure and a second pressure delivering structure, wherein said first pressure delivering structure delivers a first pressure through said first flexible tube and said second pressure delivering structure delivers a second pressure through said second flexible tube.

In these embodiments, said first pressure is either a positive pressure for delivering a fluid through said single output lumen or a negative pressure for aspirating a fluid through single output lumen and said second pressure is either a positive pressure for delivering a fluid through said single output lumen or a negative pressure for aspirating a fluid through said single output lumen.

In a preferred embodiment, the pressure delivering structure is selected from the group consisting of (1) a syringe with a sealed stopper that mechanically provides a continuous source of said positive pressure, (2) a syringe with a sealed stopper that locks to provide a continuous source of said negative pressure, (3) a tube connected to an external device that provides a continuous source of said positive pressure, and (4) a tube connected to an external device that provides a continuous source of said negative pressure.

In another preferred embodiment, the first or second pressure delivering structure is an expandable chamber. In another preferred embodiment, a needle is connected distally to a fitting on the connecting structure.

The invention contemplates that the pinching structures are controlled by sliding switches, scroll wheels, or one or more control buttons. In a preferred embodiment, the invention encompasses a method of delivering or aspirating a fluid to a tissue using the device of the invention, wherein said device is controlled by an operator's first hand and a real-time imaging technology is controlled by the operator's second hand.

In other preferred embodiments, the real-time imaging technology is a form of radiology, nuclear medicine, endoscopy, thermography, or microscopy. In preferred embodiments, the real-time imaging technology selected from the group consisting of ultrasoundography, SonixGPS™ (Ultrasonix, Richmond, British Columbia, Canada) fluoroscopy, projectional radiography, magnetic resonance imaging, use of fiduciary markers, positron emission tomography (PET), scintigraphy, SPECT imaging, photo acoustic imaging, and tomography.

The invention contemplates using the disclosed devices for drug delivery, cyst aspiration, hydro-dissection, intra-spinal aspiration or injection, muscular injection, tendon injection, intralesional injection, chemotherapy injection into or near tumors, injection or aspiration of joints or joint-related structures, deep vascular access, or vein sclerotherapy. In certain aspects, the method of using the device is for intralesional chemotherapy, intraarterial chemotherapy, vein sclerotherapy, proliferant therapy, needle biopsies, prolotherapy, or aspiration of bodily fluid. In one embodiment, the device is used for injecting a drug selected from the group consisting of an anesthetic; a steroid, a physiological solution, and a pharmaceutical composition.

In a preferred embodiment, the barrel comprises a third flexible tube and a pinching structure for alternatively releasing said first, second or third flexible tubes, said connecting structure converges the contents of said first, second and third flexible tubes into a single output lumen, and said supporting structure connects a third pressure delivering structure to said third flexible tube.

In another embodiment, the selective pinching structure within the device may perform each of the following; a) pinch said first flexible tube and release said second flexible tube thereby allowing the pressure at said second pressure delivering structure and said single output lumen to be about the same, b) release said first flexible tube and pinch said second flexible tube thereby allowing the pressure at said first pressure delivering structure and said single output lumen to be about the same, and c) pinch said first and second flexible tube thereby allowing the pressure at said single output lumen to be about neutral.

The invention contemplates a method for delivering anesthesia, the method comprising inserting a device for single-handed aspiration and injection, the device comprising; a) an axially extending barrel having a cavity comprising a first flexible tube and a second flexible tube, said tubes extending from a proximal end to a distal end, said barrel comprising a structure for selectively pinching said first and said second flexible tubes, b) a connecting structure for converging the contents of said flexible tubes into a single output lumen, wherein the connecting structure is mounted on and extending distally from said structure for selective pinching, c) a supporting structure on a proximal end for connecting a first pressure delivering structure and a second pressure delivering structure, wherein said first pressure delivering structure delivers a first pressure through said first flexible tube and said second pressure delivering structure delivers a second pressure through said second flexible tube, wherein said first pressure is either a positive pressure for delivering a fluid through said single lumen or a negative pressure for aspirating a fluid through said single lumen and said second pressure is either a positive pressure for delivering a fluid through said single lumen or a negative pressure for aspirating a fluid through said single lumen. The method of using the device may comprise controlling the device with an operator's first hand and controlling a real-time imaging technology with an operator's second hand.

In another embodiment, the invention provides for a method for single-handed sequential injection of material into a patient in need thereof, comprising: a) placing said needle of said single-handed injection device disclosed herein in proximity to an anatomical place of interest, b) injecting a first injectate into said anatomical place of interest, c) switching said selective pinching structure to pinch closed said flexible tube associated with said first injectate and to open said flexible tube associated with a second injectate, and d) injecting a second injectate into said anatomical place of interest.

A method for single-handed sequential injection and aspiration of materials into or from a patient in need thereof, comprising: a) placing the needle of said single-handed injection device disclosed herein in proximity to an anatomical place of interest, b) injecting an injectate into said anatomical place of interest, c) switching said selective pinching structure to pinch closed said flexible tube associated with said injectate and to open said flexible tube associated with a negative pressure, and d) aspirating a material from said anatomical place of interest. The aspirating step and injecting step may alternatively be performed in reverse order such that the aspirating step is accomplished prior to the injecting step.

An advantage of an embodiment of the device is that an operator can perform a real-time imaging-guided injection, such as for delivery for anesthesia, without the assistance of a second person. In this aspect, the device leaves the second hand of the operator free to control the imaging device.

Thus, in certain embodiments, the device eliminates the need for another person to inject solution or hold an ultrasonic probe. It also allows for controlled amounts of solution in small amounts to be delivered in the desired location. In addition, the device allows for improved methods of hydro-dissection of tissues.

The device also can be used for a wide variety of other tasks associated with controlling the injection of, or aspiration of, any type of fluid or material, including, by way of example, injecting and/or aspirating medicinal solutions, bodily fluids, platelet rich plasma, and anesthesia.

While the attached drawings show, describe, and point out novel features as applied to various embodiments, it will be understood that various omissions, substitutions, and changes in the form and details of the device may be made without departing from the spirit of the disclosure. As will be recognized, certain embodiments of the inventions described herein may be embodied within a form that does not provide all of the features and benefits set forth herein, as some features may be used or practiced separately from others.

### BRIEF DESCRIPTION OF THE DRAWINGS

FIG. 1 is a schematic illustration of an embodiment of the invention showing an assembled dual syringe hand held control device utilizing a sliding switch as the finger actuator.

FIG. 2 is a schematic illustration emphasizing the organization of a dual syringe hand held control device utilizing a sliding switch as the finger actuator.

FIG. 3 is a schematic illustration emphasizing the positions of pinchers in certain embodiments of a dual syringe hand held control device utilizing a sliding switch as the finger actuator.

FIG. 4 is a schematic illustration emphasizing the positions of pinchers in certain embodiments of the invention in a dual syringe system. The slider switch has an underside tapering that when engaged pushes the buttons and forces a first pincher to close a first flexible tube while a second pincher is positioned to allow for a second flexible tube to be open.

FIG. 5 is a schematic illustration emphasizing the positions of pinchers in certain embodiments of the invention and shows the device from the opposite side. The use of cantilevers below the pinchers forces the pincher positions towards the sliding switch. An underside taper on the bottom of the slider switch increasingly pushes the first button to release the lumen of the first flexible tube.

FIGS. 6a and 6b are schematic illustrations emphasizing the structural features of pinching buttons and flexible tubes and how those features may affect mechanical outcomes. Having a pinching button in a pressed state effectively opens the tubing to flow while having a pinch button in a depressed state closes the tube. The use of cantilevers below the pinchers force the pinching button positions towards the depressed state.

FIG. 7 is a schematic illustration emphasizing a system where the positioning of a sliding switch, the structural features and positioning of pinching buttons, and cantilevers may affect the flow state within flexible tubing.

FIG. 8 is a schematic illustration showing an embodiment of the invention utilizing a scroll wheel as the finger actuator and a dual syringe setup.

FIG. 9 is a schematic illustration of a scroll wheel finger actuator.

FIG. 10 is a schematic illustration showing an embodiment of the invention utilizing a dual control button finger actuator and two expandable chambers.

FIG. 11 is a schematic illustration of a dual control button finger actuator.

FIG. 12 is a schematic illustration showing an embodiment of the invention utilizing a single hand controller encasing harboring a dual line controller.

FIG. 13 is a schematic illustration of a single hand controller encasing from a frontal orientation.

FIG. 14 is a schematic illustration of a single hand controller encasing from a side orientation.

FIG. 15 is a schematic illustration showing an embodiment of the invention utilizing push/pull control buttons as the finger actuator.

### DETAILED DESCRIPTION OF THE INVENTION

The particulars shown herein are by way of example and for purposes of illustrative discussion of the invention only and are presented to provide what is believed to be the most useful and readily understood description of the principles and conceptual aspects of the invention. It will nevertheless be understood that of the scope of the disclosure invention includes equivalents not particularized herein but nonetheless enabled by the instant specification or would be known to skilled artisans.

The disclosed embodiments include devices, systems, and methods useful for delivery and/or aspiration of fluids into patients. For example, various embodiments provide for percutaneous access into a patient. In at least some embodiments, the single hand held device allows for aspiration of bodily fluid and delivery of a medicine, such as an anesthetic, via injection into regions of interest.

“Distal” means further from the point controlled by the operator (e.g., physician or technician) of a device. “Distal” may also refer to the opposite end of a structure with reference to a “proximal” end.

“Fluid” means a substance offering no permanent resistance to change of shape, such as a gas or a liquid.

“Proximal” means closer to the point controlled by the operator (e.g., physician or technician) of a device. “Proximal” may also refer to the opposite end of a structure with reference to a “distal” end.

Currently, ultrasound guided procedures require an assistant to help with the injection. The physician typically uses one hand to hold the ultrasound transducer while the other hand holds the needle or syringe for injection. As one hand is utilized to hold the ultrasound transducer and the other hand used to stabilize the needle, an assistant is required to help push the syringe plunger. Alternatively, the physician may utilize one hand to hold the syringe and to control injection while an assistant holds the ultrasound transducer. Requiring a second person is disadvantageous 1) the physician performing the injection does not have complete control of the syringe, injection actions, and transducer positioning; and 2) the second person needed to perform the procedure is not available to carry out his or her other medical office duties. In particular, for UGRA procedures, typically large volumes of anesthetic are required to be injected which may be as much as 30 cc or more. Maintaining delivery accuracy and consistency under such conditions could be enhanced if the physician could control each aspect of the procedure.

New single-handed techniques include the Jedi Grip (David Pappin, Anaesthesia, May 2011) and other single hand techniques (see, e.g. Nigel Bedforth, Anaesthesia, May 2011). These single-handed techniques allow for the control of a single syringe chamber for delivering anesthesia. Other single chamber devices for injection and aspiration procedures have been previously described (see, for example U.S. Pat. No. 7,935,092 to Odland et al., U.S. Pat. No. 8,100,865 to Spofforth, and U.S. Pat. Publ. No. 2011/0087173 to Sibbitt Jr. et al.). Certain dual chamber devices for injection and aspiration procedures have also been published (see, e.g. U.S. Pat. No. 7,959,612 to Thompson et al., U.S. Pat. No. 7,967,793 to Sibbitt Jr. et al., and U.S. Pat. Publ. No. 2012/0035532 to Meisheimer et al.). The foregoing patents and publications are incorporated by reference in their entirety. The devices in the foregoing references, however, do not provide for 1) a single hand control device that is 2) capable of injection and/or aspiration and 3) has a precision finger actuator that toggles between multiple pressure delivery structure and controls the amount of pressure being delivered 4) in a convenient grip structure.

Real-time imaging-directed needle and syringe procedures are increasingly used throughout medicine, including critical care, emergency medicine, anesthesiology, nephrology, endocrinology, and in the musculoskeletal clinic amongst others. Many sonographic procedures including anatomic hydrodissection, regional anesthesia (e.g. nerve blocks), intra-spinal space detection, suction biopsy, injection in tight joints such as the hip, and aspiration of collapsing cystic or multicystic structures, require frequent real-time adjustment of needle tip position under fine control while simultaneously aspirating or injecting with a syringe, maneuvers that can be difficult with one hand. To overcome this, devices that enable one-handed use, fine control of aspiration and injection are needed so that the second hand of an operator may be allowed to operate an ultrasound transducer or other imaging device.

Such a device may be useful in applications for applying regional anesthesia, therapeutic dosing requiring multiple injections, platelet rich plasma, intralesional chemotherapy (the injection of anticancer drugs directly into a tumor that is in the skin, under the skin, or in an organ inside the body), prolotherapy, joint and tendon injections or aspirations, injection or aspiration of joint-related structures, intra-arterial chemotherapy, vein sclerotherapy, and needle biopsies. Such a device may also be useful in general drug delivery, cyst aspiration, hydro-dissection, intra-spinal aspiration or injection, muscular injection, and deep vascular access. The invention contemplates using the disclosed device for drug delivery, cyst aspiration, hydro-dissection, intra-spinal aspiration or injection, muscular injection, deep vascular access, and vein sclerotherapy.

The invention contemplates using the disclosed devices for Intra-arterial chemotherapy. Cancerous tumors require a supply of blood and oxygen so that they can grow. They get these essentials from the arteries that supply organs with their blood and oxygen. The disclosed device can be used to deliver chemotherapy drugs into the arteries and provide access to cancerous tumors. Exemplary tumors for this embodiment include liver and brain cancers.

The invention contemplates using the disclosed devices for vein sclerotherapy. This is a medical procedure used to eliminate varicose veins and spider veins. Sclerotherapy involves an injection of a solution (generally a salt solution) directly into the vein. The solution irritates the lining of the blood vessel, causing it to swell and stick together, and the blood to clot. Over time, the vessel turns into scar tissue that fades from view.

The invention contemplates using the disclosed devices for proliferant therapies (e.g. dextrose or platelet rich plasma injection(PRP)). PRP involves the application of concentrated platelets that release growth factors to stimulate recovery in non-healing injuries. The preparation of therapeutic doses of growth factors involves an autologous blood collection (blood from the patient), plasma separation (blood is centrifuged), and application of the plasma rich in growth factors (injecting the plasma into the area.) Soft tissue injuries treated with PRP include tendinopathy, tendinosis, acute and chronic muscle strain, muscle fibrosis, ligamentous sprains and joint capsular laxity. PRP has also been utilized to treat intra-articular injuries. Examples include arthritis, arthrofibrosis, articular cartilage defects, meniscal injury, and chronic synovitis or joint inflammation.

In one embodiment, the design of the invention utilizes a hand held control device that is held between the thumb and middle finger. The device encases a Y shaped fluid channel that connects to a flow control structure, such as a finger actuator, which the operator is able to control (e.g. by using the thumb or index finger) by pressing push buttons, using a dial wheel, or a sliding switch, any of which affect the open or closed state of lumens within flexible tubes. The device allows a needle to be connected by standard connection structure, such as a luer-lock, to the distal end and having a source of positive and/or negative pressure to be connected to the proximal end. In a preferred embodiment, negative pressure syringes, such as Vacu-lok syringes (e.g. Merit Medical, South Jordan, Utah), are commercially available. The positive pressure syringe may optionally utilize an internal spring within the barrel of the syringe. The user may choose any size syringe as appropriate for the procedure for the aspiration or injection process. In certain embodiments, a dual positive pressure syringe setup may be utilized for situations requiring delivery of more than a single medicament.

Persons of skill in the art may use any commonly available source of positive or negative pressure. In one embodiment, positive pressure within the syringe may be achieved with a spring-loaded plunger containing a plunger lock. The plunger may be pushed into the syringe and locked, thereby creating a positive pressure. The device will remain with neutral pressure in the single output lumen until the actuator releases the flexible tube associated with the positive syringe thus allowing for a positive pressure to be present in the single lumen. Alternatively, a positive pressure leurlock or device connection to a compression pump may be used.

In another embodiment, a spring-loaded plunger with a plunger lock may also be used to achieve negative pressure. The plunger may be pulled out of the syringe and locked. The device will remain with neutral pressure in the single output lumen until the actuator releases the flexible tube associated with the negative syringe thus allowing for a negative pressure to be present in the single lumen. Alternatively, the device may be connected to a vacuum pump to achieve a negative pressure.

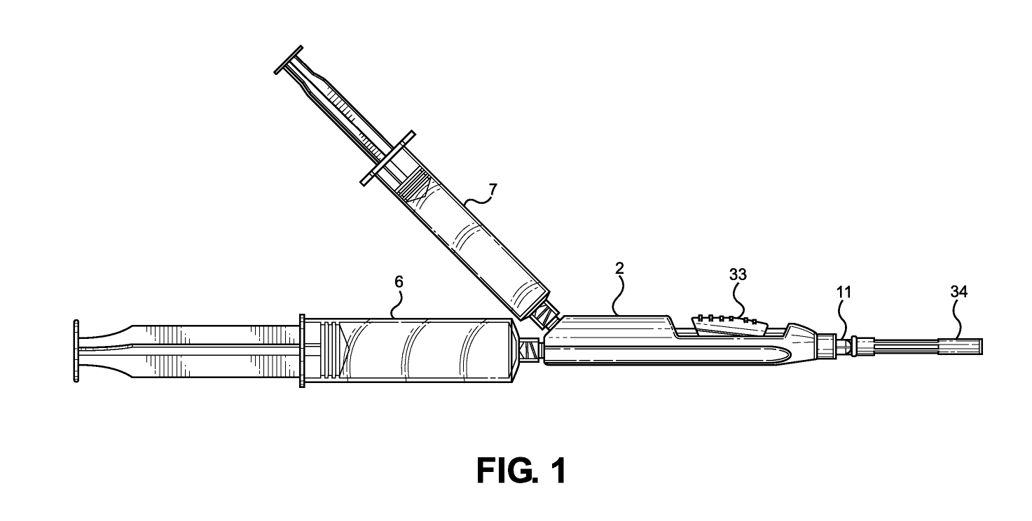

FIG. 1 is a schematic illustration showing an embodiment of the invention showing an assembled hand control actuator 2 utilizing a sliding switch 33 as the finger actuator. The device utilizes a distal connector 11 with a fitting for a needle attachment. Such needle fittings are known to those of skill in the art (e.g. a luer fitting) for attaching the needle to the hand control actuator 2. In this illustration, the needle is encased within a needle cap 34. The device is connected to two syringes 6 and 7 at the proximal end.

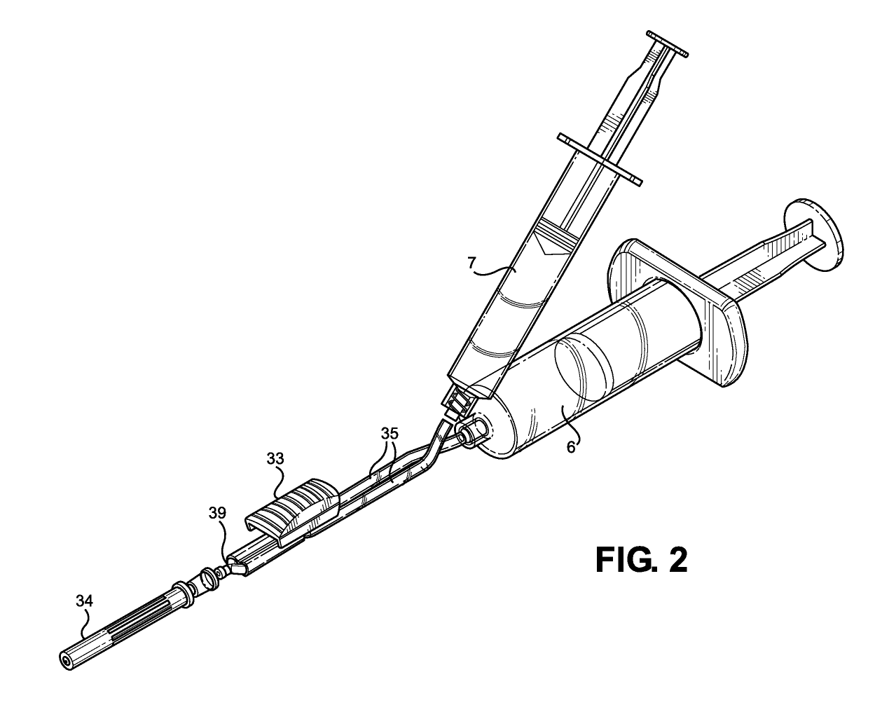

FIG. 2 is a schematic illustration showing a disassembled hand control device. The casing is removed to portray the arrangement of flexible tube 35 from each syringe 6 and 7 and into the needle cap 34. The flexible tube 35 from each syringe connects into a Y-shaped tube with a single output lumen 39 for single injection/aspiration activity through the needle.

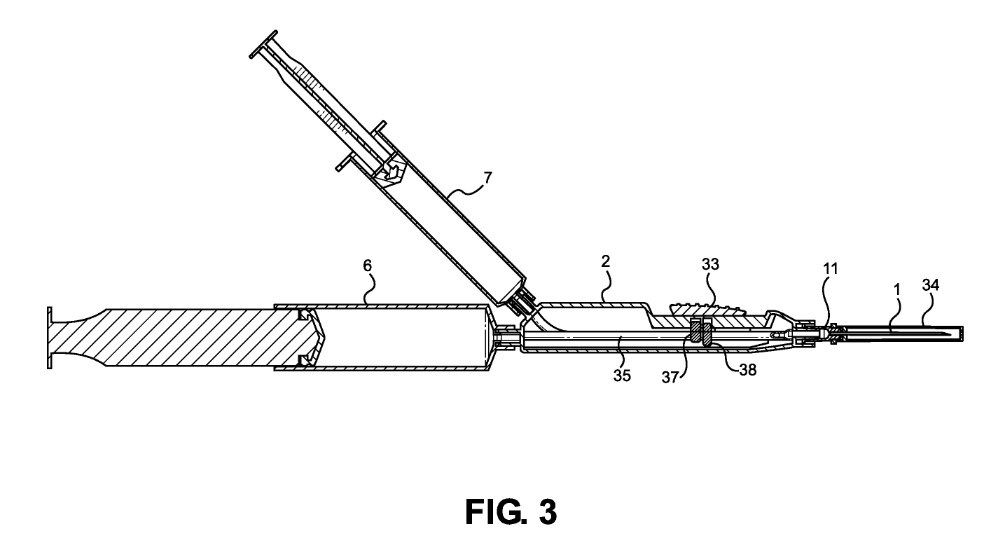

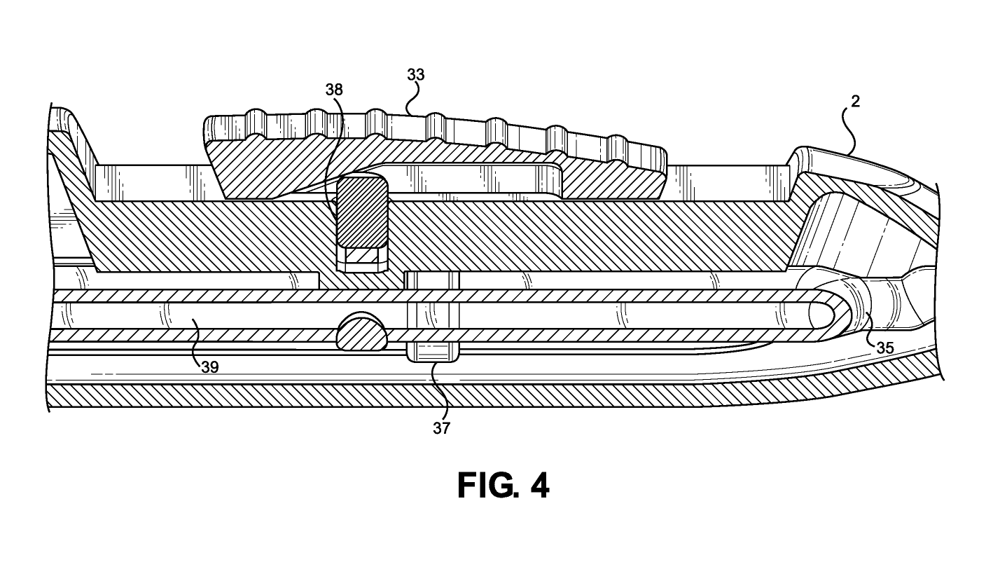

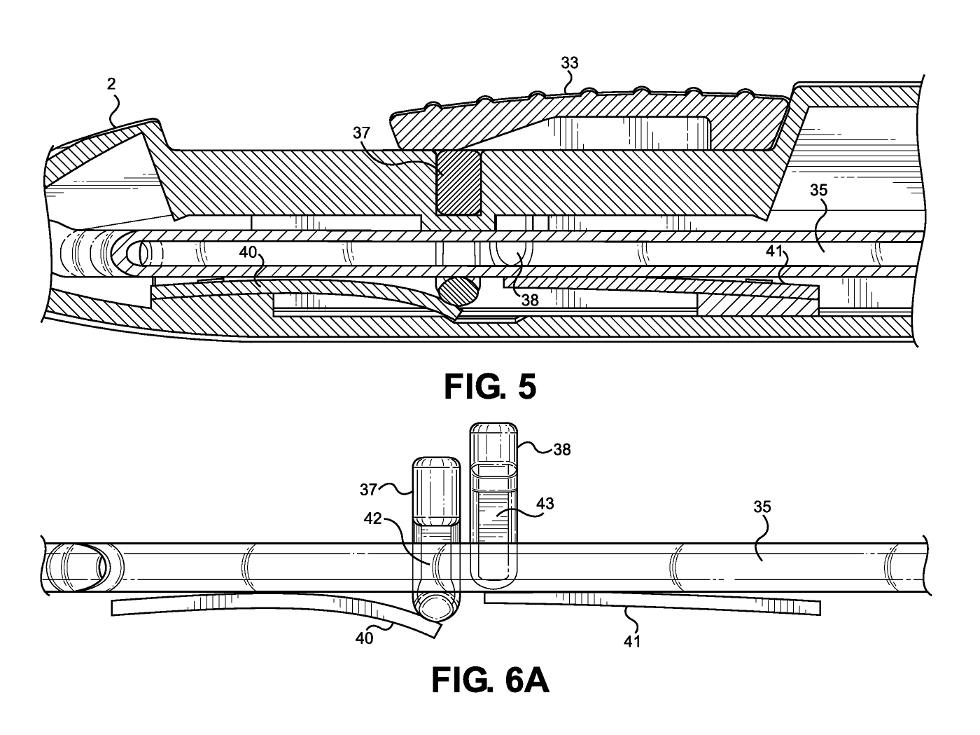

FIG. 3 shows the hand control actuator 2 from a side perspective with a transparent frame through the encasement. In this representation, the sliding switch 33 position forces pinch button 38 to be in an open state position while pinch button 37 is in the pinched position. Such an embodiment would allow for either aspiration and injection or dual injection to be performed, depending on whether an aspiration or injection structure is connected to flexible tube 35 going through pinch button 38. FIG. 4 shows a closer representation of the embodiment described in FIG. 3. FIG. 5 shows an embodiment of the invention wherein cantilever 41 forces pinch button 38 towards the sliding switch 33, effectively keeping pinch button 38 in the pinch position until an operator utilizes the sliding switch 33 to change the positioning of the pinch button to the open state. In contrast, sliding switch 33 forces pinch button 37 into the open position, which, in turn, pushes cantilever 40 downward. FIGS. 6a and 6b show the embodiment of FIG. 5 without the device encasing and further detail how flexible tubing 35 passes through the structural U-shaped turn 42 of pinch button 37 and U-shaped turn 43 of pinch button 38.

In certain embodiments, the slider button 33 is in a position where both buttons 37 and 38 are in a pinched state and obstruct any flow within the flexible tube 35. Sliding the slider button 33 distally allows button 38 to be pushed down and thus opens the flexible tube 35 while button 37 remains in the pinched state and obstructs the flexible tubing 35 within its U-shaped turn 43. Similarly, when the slider button is slid proximally, button 38 remains in the pinched state while button 37 is allowed to open the lumen of the flexible tubing 35 within its U-shaped turn 42. In certain embodiments, a spring may be used to reset the slider button 33 to the neutral position, thus pinching both pinch buttons 37 and 38 to a closed state, when force is not exerted upon it.

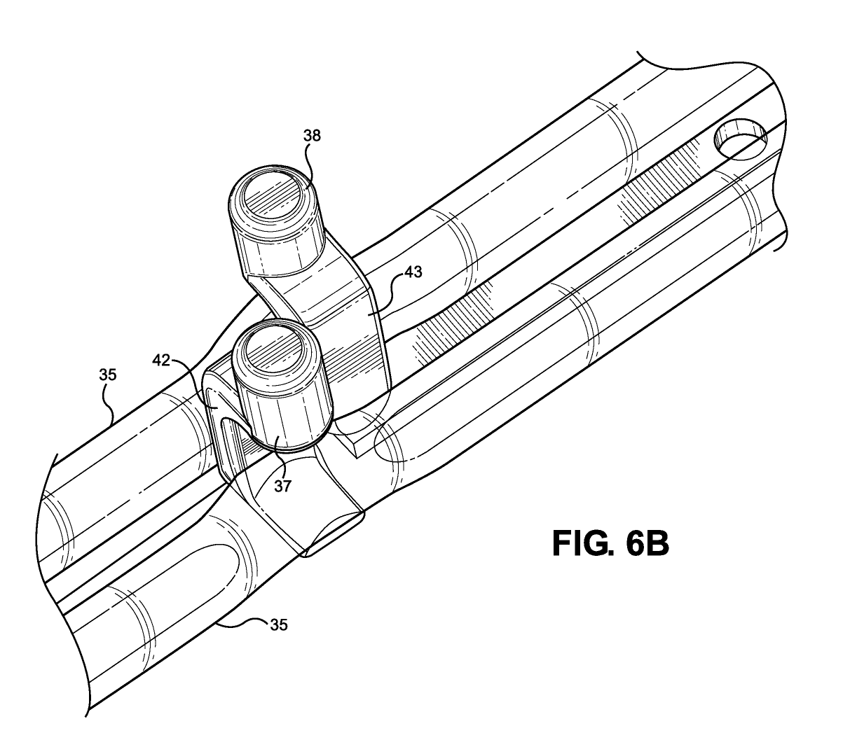

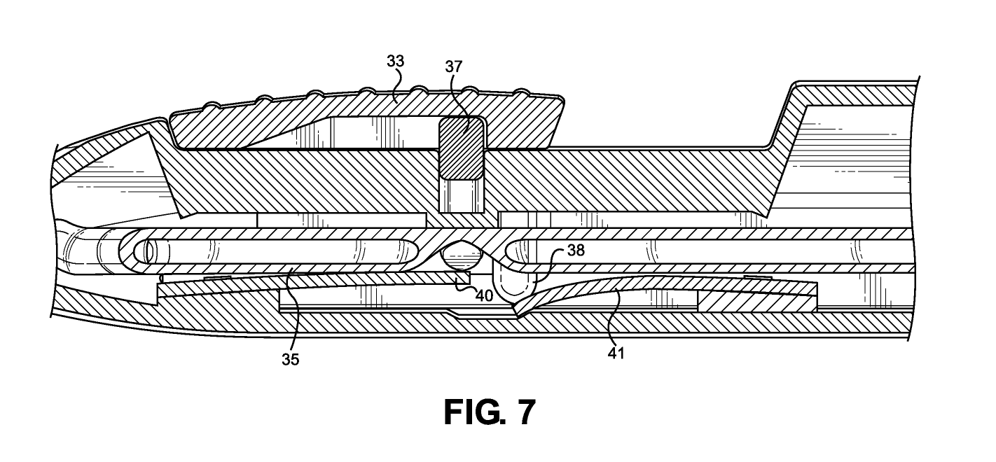

FIG. 7 shows an embodiment of the hand control device wherein the slider button 33 is in the farthest distal position. This positioning puts pinch button 38 into the open state while pinch button 37 is in the pinch state.

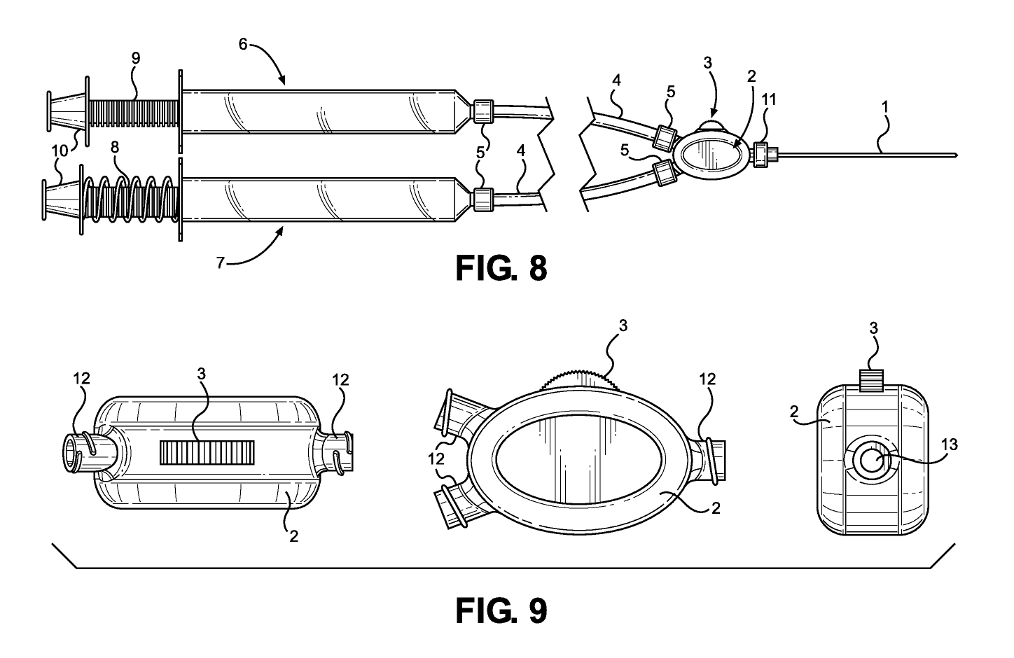

FIG. 8 is a schematic illustration showing an embodiment of the invention utilizing a scroll wheel 3 on the hand control actuator 2. Needle 1 attaches to the hand control device 2 via needle fitting 11. In certain embodiments, fitting 11 may be a luer lock. Tubing 4 attach to both the hand control actuator 2 and syringes 6 and 7 by connectors 5. In this embodiment, syringe 6 is an injector syringe while syringe 7 is an aspirator syringe containing a spring-loaded plunger 8. Syringe 6 uses plunger 9 for injection, which may be induced by pressing on the thumb rest 10.

FIG. 9 shows further details of the hand control actuator with a scroll wheel 2. At either end are attachment structures 12 for attaching tubes 4 or needle fitting 11. In the embodiment illustrated, the attachment structures 12 support screw type attachments. The lumen 13 within the hand control actuator 2 can optionally house flexible tubing 35.

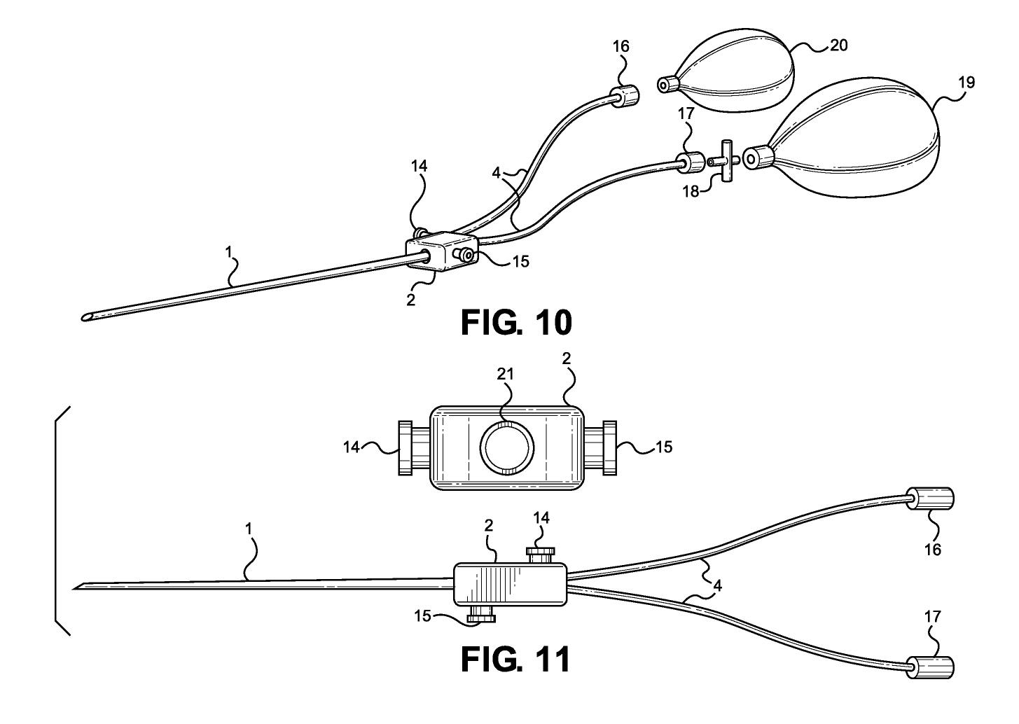

FIG. 10 shows an embodiment of the single hand control device wherein external pinch buttons 14 and 15 are utilized on the hand control actuator 2. In this embodiment, attaching tubes 4 are connected to an aspirator chamber 20 through connection 16 and an expandable chamber 19 through connector 17. A stopcock 18 may be further used to control the flow of material from the expandable chamber 19. In this particular embodiment, external pinch button 14 controls the aspiration while external pinch button 15 controls the injection. FIG. 11 shows a side view of the embodiment described in FIG. 10. Lumen 21 of the hand control actuator 2 houses flexible tubing 35 similarly to embodiments described previously (e.g. see FIGS. 6a and 6b).

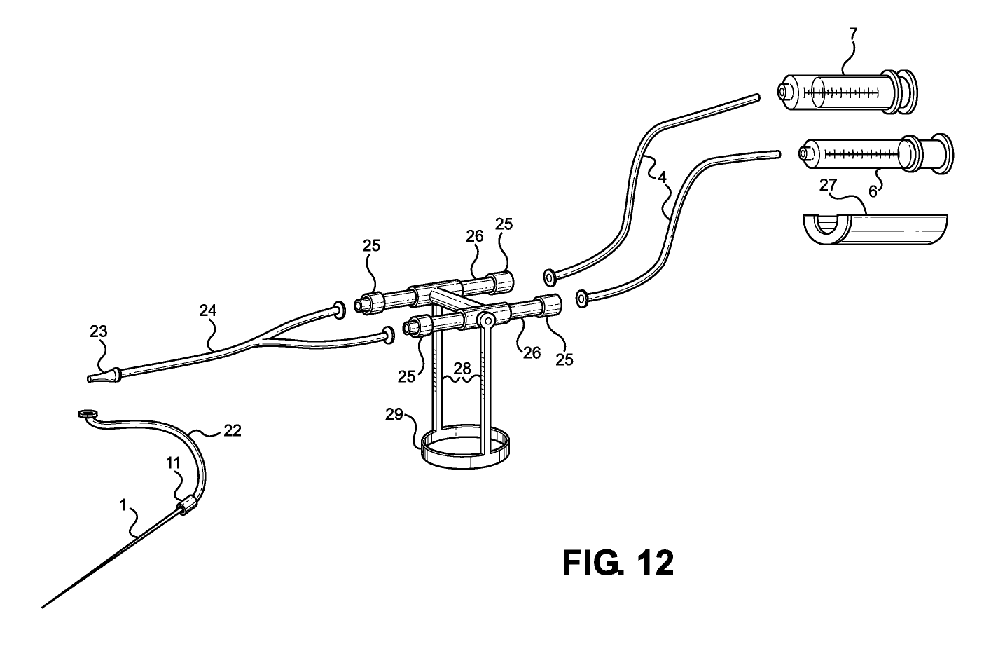

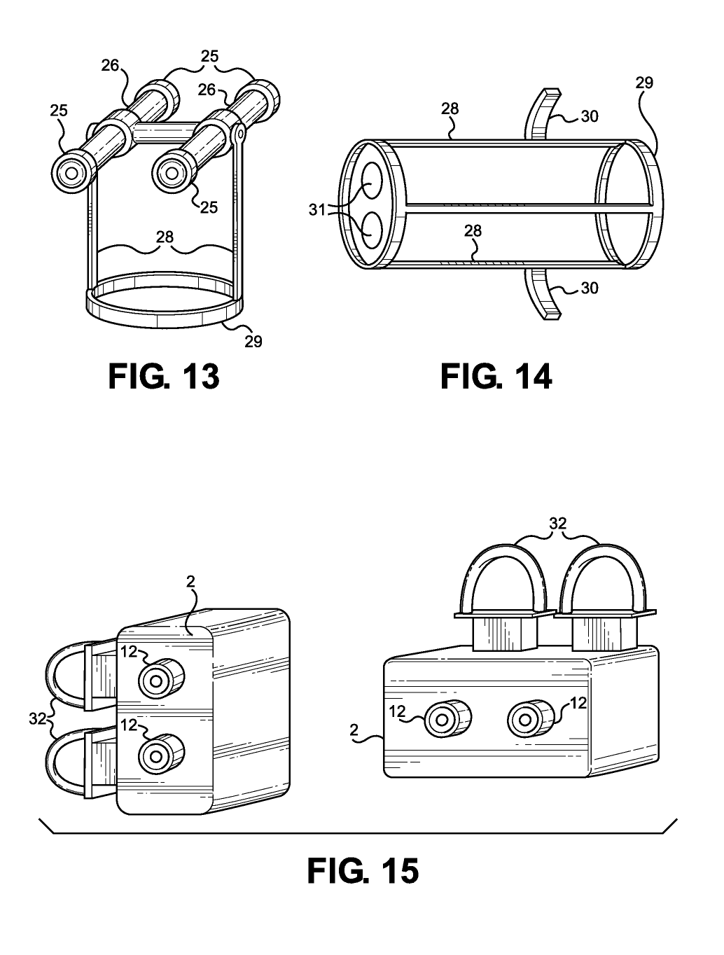

FIG. 12 shows an embodiment of the single hand control device wherein an arm casing is utilized. The arm casing comprises an approximately circular structure 29 or other ergonomic shape where a users arm may be inserted. Support beams 28 connect the circular structure 29 to injection barrels 26. Tube connectors 25 are present on both ends of the injection barrels 26. A Y-tube 24 connects on the distal end of the device that subsequentially connects to a needle tube 22 and a needle 1. Attaching tubes 4 are connected to the proximal end of the injection barrels 26. In one aspect, the attaching tubes 4 connect to syringes 6 and 7. In one aspect, syringe 6 is an injector syringe. In one aspect, syringe 6 utilizes an auto injector 27 to deliver its contents. In one aspect, syringe 7 is an aspiration syringe. FIG. 13 shows a front view of the single hand control device embodiment described in FIG. 12. FIG. 14 shows a side view of the single hand control device embodiment described in FIG. 12. A control element as disclosed herein is used to choose the pressure delivered by one of tubes 4.

FIG. 15 shows an embodiment of the single hand control device wherein trigger finger actuators 32 are utilized to control the hand control actuator 2. Attaching tubes 4 connect to the hand control actuator 2 at lumens 12. The trigger finger actuators 32 contain circular rings where a user may slide their finger into the ring portion of the finger actuator for push/pull engagement. This embodiment allows for rigid control of the aspiration and/or injection functions.

For suction biopsy procedures, the control switch is placed in a position where all flexible tubes are closed. The plunger is then pulled back to create vacuum in the syringe and the plunger may be rotated to lock the plunger in the aspiration position. The needle is placed in the body at an anatomically desired position, and then the actuator switch is moved to open the flexible tube lumen associated with the vacuum set syringe and the fluid aspiration or biopsy is performed. Once done with the aspiration procedure, the actuator switch is moved to a position that closes the associated flexible tubing, and the needle extracted. To expel the sample, the control switch is positioned to permit pressure to flow into the barrel from the injector syringe. In certain embodiments, the operator may manually push the plunger on the injector syringe to expel. In other embodiments, the injector syringe is associated with an automatic injector which forces injectate into the barrel. In certain embodiments, dual sequential injections are needed. For these procedures, once the first injectate has been delivered up to an aspired amount, the control switch position may be changed by the operator to close off the flexible tubing associated with the first injectate and open the flexible tubing associated with a second injectate and the process for expulsion is performed as needed.

For regional anesthesia administration, hydrodissection procedures, dilation of musculoskeletal structures, or to autodetect the intra-spinal space, the injector syringe is first filled with fluid, typically saline or local anesthetic. The aspiration and injector syringes may be prepared as described above. The needle is then inserted in the anatomic target of interest. When the control switch is positioned to open the flexible tube associated with the injector syringe, the syringe automatically injects. Flow rate is controlled by needle length and diameter and by the operator, who can move the control switch incrementally to variable proximal or distal positions for flow control facilitation. In the case of nerve blocks the automatic syringe injects local anesthetic under great control next to the target nerve. The aspiration function is used variably as the needle is positioned to the point of anatomical interest. The control button closes the injector syringe associated flexible tube while opening up the aspiration associated flexible tube. Sample aspirations are taken and blood uptake monitored to ensure no capillary, vein, or artery piercings occur. A lack of blood update confirms no piercing. Once the needle approaches the point of interest, the control switch is again moved, this time to close the aspiration associated flexible tube and open up the injector associated flexible tube. For hydro-dissection the operator uses the automatic syringe to dissect anatomic planes and free trapped structures under image guidance. In the case of musculoskeletal injections, the target structure is dilated to create a space for corticosteroid, hyaluron, or other agents. After dilation, a flow exchange from a first syringe to a second syringe may be performed without needle movement.

In certain aspects, devices of the present invention are compatible with certain vacuum syringes having the ability to lock or otherwise restrain the plunger and to close the flow switch. This permits a vacuum (i.e. negative pressure) to reside in the syringe in a stable, secure fashion and permits precise release of the vacuum for fluid aspiration or suction biopsy procedures. Vacuum syringes are used for amniocentesis, liposuction, fat biopsy, vacuum curettage, and fine-needle aspiration biopsy.

A further advantage of the present invention is the ability to continue work during instances of needle clogging during aspiration. If the needle becomes clogged, it may be cleared by switching to the injection syringe and expelling the clog. Aspiration may then be resumed by gently switching back to the aspiration setup.

Plunger locks are used in conventional syringes to prevent unintended injection or loss of fluid contained within a syringe, or alternatively to maintain pressure or vacuum in a syringe.

Certain medical procedures require the administration of two or more treatment agents to a desired location within the body of a patient such that the substances become combined or mixed shortly before, during or shortly after delivery into the body. For example, some therapies involve the administration of two or more component substances (e.g., chemical compounds, solutions, suspensions, biologics, cells, reactants) such that those substances react or otherwise interact with each other to form a resultant mixture or reaction product that directly or indirectly results in some therapeutic, diagnostic or cosmetic benefit (generally referred to herein as “Multiple-Component Therapies”). In some cases, it is important for the component substances to be combined or mixed at precise location and volumes immediately before, during or after delivery. For example, mixing or combining of component substances too long before delivery may result in increased viscosity or a solidification process may occur that would render the product incapable of passing through an intended delivery structure, such as a needle, or where the product has a very short half life and would loose activity before reaching its intended in vivo destination.

In certain embodiments, the invention contemplates delivering pharmaceutical compositions. Non-limiting examples of such compositions include chemotherapeutic agents (e.g. Bleomycin, 5-FU, Vinblastine, Doxorubicin, and Vincristine), anticoagulants, vasodialators, kinase modulators, steroid anti-inflammatory agents, non-steroid anti-inflammatory agents, proliferant agents (e.g. dextrose, glycerin, zinc, calcium, manganese, phenol, guaiacol, tannic acid, plasma QU, pumice flour or other particulates, sodium morrhuate, and growth factors), and neurolytic agents (e.g. alcohol, phenol, and glycerol).

In other embodiments, the invention contemplates delivering anesthetic compositions. Non-limiting examples of such compositions include Lidocaine, Bupavicaine, Mepivacaine, Ropivacaine, Chlorprocaine, Levobupivacaine, Epinephrine, Clonidine, Prilocaine, Etidocaine, and opioids (e.g. Morphine and Fentanyl).

One Multiple Component Therapy known in the art is platelet gel, or PG. In this therapy, a platelet-containing component (e.g. platelet rich plasma (PRP)) is combined with a thrombin-containing component (e.g. a thrombin solution) immediately before, during or after injection into the myocardium at an anatomical location of interest within or near an infarct or other myocardial injury. The platelet-containing component combines with the thrombin-containing component and forms a platelet gel that causes the desired therapeutic effect. Such PG is formed when components (such as fibrinogen) contained in the platelet-containing component are activated by thrombin contained in the thrombin-containing component. The addition of thrombin to platelet-containing plasma products such as PRP is described in detail in U.S. Pat. No. 6,444,228, to Baugh et al., incorporated by reference herein in its entirety. PRP is also used in a variety of orthopedic and other applications.

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. A device for single-handed aspiration and injection, comprising:
a. an axially extending barrel having a cavity comprising a first flexible tube and a second flexible tube, said tubes extending from a proximal end to a distal end, said barrel comprising a structure for selectively pinching said first and said second flexible tubes,
b. a connecting structure for converging the contents of said flexible tubes into a proximal end of a single output lumen, wherein the connecting structure is mounted on and extending distally from said barrel and wherein a fitting for a hypodermic needle is connected to a distal end of said distal connector, and
c. a supporting structure on the proximal end of said barrel for connecting a first pressure delivering structure and a second pressure delivering structure, wherein said first pressure delivering structure delivers a first pressure through said first flexible tube and said second pressure delivering structure delivers a second pressure through said second flexible tube,
wherein said first pressure is either a positive pressure for delivering a fluid through said single output lumen or a negative pressure for aspirating a fluid through single output lumen and said second pressure is either a positive pressure for delivering a fluid through said single output lumen or a negative pressure for aspirating a fluid through said single output lumen.

2. The device of claim 1, wherein said first or said second pressure delivering structure is selected from the group consisting of (1) a syringe with a sealed stopper that mechanically provides a continuous source of said positive pressure, (2) a syringe with a sealed stopper that locks to provide a continuous source of said negative pressure, (3) a tube connected to an external device that provides a continuous source of said positive pressure, and (4) a tube connected to an external device that provides a continuous source of said negative pressure.

3. The device of claim 2, wherein a needle is connected to said needle fitting.

4. The device of claim 1, wherein said barrel comprises a third flexible tube and a pinching structure for alternatively releasing said first, second or third flexible tubes, said connecting structure converges the contents of said first, second and third flexible tubes into said single output lumen, and said supporting structure connects a third pressure delivering structure to said third flexible tube.

5. The device of claim 1, wherein said pinching structure is controlled by a sliding switch, scroll wheel, or one or more control buttons.

6. The device of claim 5, wherein said pinching structure is a sliding switch.

7. A method of delivering or aspirating a fluid to a tissue using the device of claim 1, wherein said device is controlled by an operator's first hand and a real-time imaging technology is controlled by said operator's second hand.

8. The method of claim 7, wherein said real-time imaging technology is selected from the group consisting of ultrasoundography, SonixGPS™, fluoroscopy, projectional radiography, magnetic resonance imaging, use of fiduciary markers, positron emission tomography (PET), scintigraphy, SPECT imaging, photo acoustic imaging, and tomography.

9. The method of claim 8, wherein said real-time imaging technology is ultrasoundography.

10. The method of claim 7, wherein said device is used for a procedure selected from the group consisting of drug delivery, cyst aspiration, biopsy aspiration, hydro-dissection, intra-spinal aspiration or injection, muscular injection, tendon injection, intralesional injection, chemotherapy injection into or near tumors, injection or aspiration of joints or joint-related structures, deep vascular access, and vein sclerotherapy.

11. The device of claim 1, wherein said structure for selective pinching may perform each of the following;
a. pinch said first flexible tube and release said second flexible tube thereby allowing the pressure at said second pressure delivering structure and said single output lumen to be about the same,
b. release said first flexible tube and pinch said second flexible tube thereby allowing the pressure at said first pressure delivering structure and said single output lumen to be about the same, and
c. pinch said first and second flexible tube thereby allowing the pressure at said single output lumen to be about neutral.

12. The method of claim 7, wherein said device is used for anaesthetic delivery, intralesional chemotherapeutic delivery, intraarterial chemotherapeutic delivery, vein sclerotherapy, proliferant therapy, needle biopsies, prolotherapy, or aspiration of bodily fluids.

13. The method of claim 10, wherein said drug is selected from the group consisting
a. an anesthetic;
b. a steroid
c. a physiological solution; and
c. a pharmaceutical composition.

14. The device of claim 1, wherein said first or second pressure delivering structure is an expandable chamber.

15. A method for delivering anesthesia, the method comprising inserting a device for single-handed aspiration and injection, the device comprising:
a. an axially extending barrel having a cavity comprising a first flexible tube and a second flexible tube, said tubes extending from a proximal end to a distal end, said barrel comprising a structure for selectively pinching said first and said second flexible tubes,
d. a connecting structure for converging the contents of said flexible tubes into a proximal end of a single output lumen, wherein the connecting structure is mounted on and extending distally from said barrel and wherein a fitting for a hypodermic needle is connected to a distal end of said distal connector, and
e. a supporting structure on a proximal end of said barrel for connecting a first pressure delivering structure and a second pressure delivering structure, wherein said first pressure delivering structure delivers a first pressure through said first flexible tube and said second pressure delivering structure delivers a second pressure through said second flexible tube,
wherein said first pressure is either a positive pressure for delivering a fluid through said single output lumen or a negative pressure for aspirating a fluid through said single output lumen and said second pressure is either a positive pressure for delivering a fluid through said single output lumen or a negative pressure for aspirating a fluid through said single output lumen.

16. The method of claim 15, wherein said device is controlled by an operator's first hand and a real-time imaging technology is controlled by an operator's second hand.

17. A method for single-handed sequential injection of materials into a patient in need thereof, comprising:
a. placing the needle of the device in claim 3 in proximity to an anatomical place of interest,
b. injecting a first injectate into the anatomical place of interest,
c. switching said selective pinching structure to pinch closed said flexible tube associated with said first injectate and to open said flexible tube associated with a second injectate, and
d. injecting a second injectate into said anatomical place of interest.

18. A method for single-handed sequential injection and aspiration of materials into or from a patient in need thereof, comprising:
a. placing the needle of the device in claim 3 in proximity to an anatomical place of interest,
b. injecting an injectate into the anatomical place of interest,
c. switching said selective pinching structure to pinch closed said flexible tube associated with said injectate and to open said flexible tube associated with said negative pressure, and
d. aspirating a material from said anatomical place of interest,
wherein said aspirating step and injecting step may alternatively be performed in reverse order such that the aspirating step is accomplished prior to the injecting step.

19. The method of claim 13, wherein said anesthetic is selected from the group consisting of Lidocaine, Bupavicaine, Mepivacaine, Ropivacaine, Chlorprocaine, Levobupivacaine, Epinephrine, Clonidine, Prilocaine, Etidocaine, and an opioid.

20. The method of claim 13, wherein said pharmaceutical composition is selected from the group consisting of a chemotherapeutic agent, an anticoagulant, a vasodialator, a kinase modulator, a steroid anti-inflammatory agent, a non-steroid anti-inflammatory agent, a proliferant agent, and a neurolytic agent.
